(19) United States
(12) Patent Application Publication
(10) Pub. No.: US 2009/0131271 A1
(43) Pub. Date: May 21, 2009

(54) METHOD FOR DELIVERING NUCLEIC ACID AND DEVICE FOR DELIVERING NUCLEIC ACID

(75) Inventors: Takuro Niidome, Fukuoka (JP); Fumio Wada, Kumamoto (JP); Yoshiki Katayama, Fukuoka (JP); Kosuke Yakumaru, Fukuoka (JP); Takahito Kawano, Fukuoka (JP); Shuzo Yamashita, Okayama (JP)

Correspondence Address: HAHN & VOIGHT PLLC, 1012 14TH STREET, NW, SUITE 620, WASHINGTON, DC, 20005, US

(73) Assignees: KYUSHU UNIVERSITY, NATIONAL UNIVERSITY CORPORATION, Fukuoka (JP); JAPAN STENT TECHNOLOGY CO, LTD., Okayama (JP)

(21) Appl. No.: 11/914,702

(22) PCT Filed: May 16, 2006
(86) PCT No.: PCT/JP2006/310100

§ 371 Date: Jan. 18, 2008

(30) Foreign Application Priority Data
May 17, 2005 (JP) JP 2005-144571

(51) Int. Cl. C40B 30/06; C12N 15/87; C40B 60/12; C12M 1/00

(52) U.S. Cl. 506/10; 435/455; 435/285.1; 506/39

(57) ABSTRACT

In a method for delivering a nucleic acid of the invention, a nucleic acid is introduced into a cell by pressing the nucleic acid supported on a surface of a solid substrate against the cell. According to the method, the nucleic acid can be delivered into the cell simply at a low cost without placing a heavy burden on the cell at a high nucleic acid delivery efficiency. By allowing the surface of the solid substrate to support the nucleic acid in the form of a complex with a polyamine or a cationic lipid and pressing it against the cell, the introduction efficiency into the cell can be further improved. In the invention, by using a nucleic acid useful for gene therapy as the nucleic acid, a high-efficiency device for gene therapy can be obtained.

### FIELD OF THE INVENTION

The present invention relates to a method for delivering (introducing) a nucleic acid into cells and a device for implementing the method. More specifically, the invention relates to a method for delivering a nucleic acid into cells for the purpose of therapy, diagnosis and molecular biological operation, and a device for delivering a nucleic acid.

### BACKGROUND ART

As methods for introducing low molecular functional nucleic acids into the cells in order to control a gene in the cells or to control the expression of a gene in the cells, there have heretofore been known a physical method such as electroporation and chemical methods utilizing a basic lipid and a basic polymer. However, these physical and chemical methods cause a serious damage to the cells and require special devices and reagents which are expensive. Therefore, it has been desired to develop a technology for delivering a nucleic acid into cells, which is inexpensive and is easy to use, exerting less burden on the cells.

As such nucleic acid delivery technologies, patent documents 1 and 2 disclose methods wherein a mixture of a gelatin and a DNA is spotted on a glass substrate, cells are sown therein so as to be cultured, and the DNA is spontaneously taken in by the cultured cells on the glass substrate. Patent documents 3 and 4 disclose devices for introducing genes by utilizing the above principle.

A patent document 5 proposes a method which is developed from the methods of the patent documents 1 and 2, and wherein a DNA and a complex of a basic lipid and the DNA are alternately laminated on a glass substrate which is treated to be hydrophilic, and the cultured cells are sown on the laminate so that the DNA is taken in by the cells.
Patent document 1: U.S. Pat. No. 6,544,790
Patent document 2: Junaid Ziauddin, David M. Sabatini, Nature, Vol. 411, 107-110 (2001)
Patent document 3: U.S. Pat. No. 6,670,129
Patent document 4: U.S. Pat. No. 6,652,878
Patent document 5: Fumio Yamauchi, Koichi Kato, Hiroo Iwata, Biochemica et Byophysica Acta Vol. 1672, 138-147 (2004)

It can be expected that all of the above methods disclosed in the above documents can be developed to become capable of spotting various kinds of genes and analyzing the functions in the cultured cells. However, they are concerned with the devices for analyzing the functions and cannot be adapted to animal organs.

### DISCLOSURE OF THE INVENTION

It has been strongly expected to succeed in the nucleic acid delivery technology based on the above-mentioned physical or chemical methods which involve problems as described above without, however, attaining any big breakthrough. The technology for delivering nucleic acid must satisfy three requirements, i.e., inexpensive and easy to use, exerts a small burden on the cells, and a high nucleic acid delivering efficiency. Building a nucleic acid delivery technology for simultaneously realizing the three requirements maintaining a high level leads to developing a basic technology that can be generally and widely applied to a gene therapy and a molecular biology.

It is therefore an object of the present invention to provide a method for delivering a nucleic acid and a device for delivering a nucleic acid (e.g., a device for gene therapy carrying a nucleic acid) satisfying the three requirements of inexpensive and easy to use, exerts a small burden on the cells and a high nucleic acid delivering efficiency.

The present inventors have conducted a keen study in order to achieve the above object, and have discovered that when a nucleic acid carried on the surface of a solid substrate is pressed onto the cultured cells, the nucleic acid can be taken in by the cells even if the nucleic acid pertains to those kinds which cannot be taken in by the cells despite it is simply brought into contact with the cells. The inventors have further discovered that when a complex of a nucleic acid and a cationic lipid or a polyamine is carried on the surface of the solid substrate and is pressed onto the cells, the nucleic acid is more efficiently taken in by the cells than when the nucleic acid alone is carried and is pressed or than when the complex is simply brought into contact with the cells. The present invention was accomplished based on the above new discovery.

According to the present invention, there is provided a method for delivering a nucleic acid by pressing the nucleic acid carried on the surface of a solid substrate onto cells to thereby introduce the nucleic acid into the cells.

In the above method for delivering a nucleic acid, it is desired that:

(1) The nucleic acid is forming a complex thereof with a cationic lipid or a polyamine;
(2) The nucleic acid is forming a complex thereof with a protamine or a dendritic polylysine; and
(3) The nucleic acid is a nucleic acid useful for a gene therapy.

The method for delivering a nucleic acid of the present invention is not only capable of efficiently delivering the nucleic acid into the cells but also features good reproducibility and can be easily operated. When the solid substrate carrying the nucleic acid is pressed onto the cells, stress is temporarily given to the cells. After the solid substrate is no longer pressed, however, basically no chemical substance is left after the treatment and a smaller burden is given to the cells than that of the prior chemical methods. Therefore, the method for delivering a nucleic acid of the invention can be applied not only to procaryotic cells such as bacteria and eucaryotic cells separated from plants and animals but also to the cells present in the tissues of plants and animals.

According to the present invention, there is further provided a device for delivering a nucleic acid, in which, the nucleic acid to be introduced into the cells is carried on a surface of a solid substrate, and the surface of the solid substrate carrying the nucleic acid is pressed onto the cells, so that the nucleic acid is introduced into the cells.

In the device of the invention, it is desired that:

(4) The surface of the solid substrate is modified with a cationic functional group;
(5) The surface of the solid substrate is modified with an anionic functional group, and the nucleic acid carried on the surface modified with the anionic functional group is forming a complex thereof with a cationic lipid or a polyamine;
(6) The nucleic acid carried on the surface of the solid substrate is forming a complex thereof with a protamine or a dendritic polylysine;
(7) The nucleic acid is a nucleic acid useful for a gene therapy; and
(8) The nucleic acid is carried in an amount of 20 nanograms to 10 micrograms per 1 cm².

The device for delivering a nucleic acid of the invention can be inexpensively and easily fabricated. By simply pressing the surface carrying the nucleic acid onto the cells, the nucleic acid can be efficiently delivered into the cells. In this case, if the nucleic acid is adsorbed and carried on the surface of the solid substrate in the form of a complex with a cationic lipid or a polyamine (hereinafter often abbreviated as “nucleic acid complex”), the cationic lipid or the polyamine works to deliver the nucleic acid into the cells more highly efficiently owing to the endocytosis mechanism. Upon modifying the surface of the solid substrate with the cationic functional group or with the anionic functional group, further, a bonding force of the nucleic acid or the nucleic acid complex to the surface of the solid substrate increases and, as a result, the nucleic acid is delivered maintaining stability.

The device for delivering a nucleic acid of the invention can be put into practical use as a device for a gene therapy by carrying a nucleic acid which is useful for the gene therapy or as a device for introducing a gene for the screening of gene expressions or for the analysis of gene functions by spotting (arranging like dots) a plurality of known genes or unknown genes.

More concretely, the device for delivering the nucleic acid of the invention can be put into practical use as:

(a) A device which carries a nucleic acid for a gene therapy and introduces the gene into the cells by pressing them onto an organ in the body;
(b) A device for executing a gene therapy by carrying a nucleic acid for a gene therapy, by introducing the nucleic acid (gene) into the cells by pressing them onto the cells taken out of the body, and by returning the cells back into the body; and
(c) A device for screening the gene expressions by spotting a plurality of known genes or unknown genes on a solid substrate, and pressing them onto the cultured cells or onto an organ of an animal, or a device for introducing the gene for the analysis of gene functions.

As a concrete example of the above device (a) for introducing a gene into the cells, there can be used a device in which the nucleic acid for the gene therapy is carried on the surface of a stent which is introduced into a body cavity such as a blood vessel to expand the structured part as well as to introduce the gene into the cells of the same part (i.e., a stent carrying on the surface thereof a nucleic acid for the gene therapy).

According to the present invention, there is provided a screening method by utilizing the above method for delivering a nucleic acid or the device for delivering a nucleic acid, for example, a method for screening genes by spotting a plurality of known genes or unknown genes on the surface of the solid substrate, pressing the spotted known genes or unknown genes onto the cultured cells or onto the organ of an animal, and screening the expressions of the genes.

According to the present invention, further, there is provided a device for introducing a gene by spotting a plurality of known genes or unknown genes onto a solid substrate, pressing the spotted known genes or unknown genes onto the cultivated cells or onto the organ of an animal to screen the expressions of the genes or to analyze the functions of the genes.

According to the present invention, there is further provided a device for a gene therapy comprising the device for delivering a nucleic acid.

### BRIEF DESCRIPTION OF THE DRAWINGS

FIG. 1 is a graph showing transfection efficiencies from the SEAM-F coated substrates carrying a nucleic acid of the invention into the CHO cells;

FIG. 2 is a graph showing transfection efficiencies from the glass substrates carrying protamine-plasmid DNA complexes of different mixing ratios into the CHO cells;

FIG. 3 is a graph showing transfection efficiencies from the glass substrates adsorbing protamine-plasmid DNA complexes (C/A ratio=4) having different DNA amounts into the CHO cells; and

FIG. 4 is a graph showing transfection efficiencies from the glass substrates adsorbing plasmid DNA complexes of different concentrations into the CHO cells.

### BEST MODE FOR CARRYING OUT THE INVENTION

### Solid Substrates

In the present invention, there is no particular limitation on the solid substrate for carrying a nucleic acid provided it is capable of carrying the nucleic acid, and any material or the solid substrate of any shape can be selectively used depending upon the object of using the device for delivering the nucleic acid and the environment in which it is used. For example, there can be used a solid substrate made of a glass, various ceramics, a metal, or natural or synthetic high molecules. Further, the solid substrate may have any shape such as a plate, a sheet, a film, a rod, a hollow cylinder, a cord, a line, a mesh structure, a woven fabric or a nonwoven fabric. Any solid substrate capable of easily carrying a required amount of nucleic acid on the surface thereof and having a shape which can be easily pressed onto the cells, may be selectively used depending upon the object of use and the mode of use.

As required, the solid substrate may be coated with a variety of polymers. When the solid substrate made of a glass is used, in particular, it is desired that the surfaces thereof are coated with a polymer. As the polymer, there can be exemplified living body-fitting polymers having a micro domain structure, such as a hydroxyethyl methacrylate/styrene block copolymer, a polyether/nylon 6-10 block copolymer and a polyether polyurethane; polymers widely used in medical field, such as polypropylene, polyethylene, polyurethane, polysulfon and nylon; and polymers thereof to which an anionic functional group is introduced.

Among the above living body-fitting polymers, the one called SEAM-F is particularly suited. The SEAM-F is a copolymer material having hydrophilic and hydrophobic micro domain structures, and is produced by blending a pentacopolymer of trimethoxysilylpropyl methacrylate (TMSPMA)/methyl methacrylate (MMA)/butyl methacrylate (BMA)/2-hydroxymethylmethacrylate (HEMA)/styrene (St), and a polyurethane having trimethoxysilyl groups at both terminals thereof, and a tetracopolymer of TMSPMA/MMA/perfluorodecylethyl methacrylate (PFDEMA)/St. The above SEAM-F has been described in, for example, K. Kawahito et al., Artificial Organs, 19 (8), 857-863 as a FASUS copolymer.

Further, the solid substrate may have its surface for carrying the nucleic acid chemically modified within a range in which it does not impair the object, action and effect of the invention. That is, upon chemically modifying the surface of the solid substrate, the bonding force can be increased between the surface of the solid substrate and the nucleic acid or a complex of the nucleic acid and the polyamine. In particular, the modification with the cationic functional group is effective in increasing the bonding force to the nucleic acid, and the modification with the anionic functional group is effective in increasing the bonding force to the complex of the nucleic acid and the polyamine. The modification with the cationic functional group can be achieved by coating the surface of the solid substrate with a material having the cationic functional group such as amino group or guanidium group. On the other hand, the modification with the anionic functional group is achieved by introducing the anionic functional group such as carboxyl group, phosphoric acid group or sulfuric acid group into the surface of the solid substrate or by coating the surface of the solid substrate with a material having the anionic functional group.

### (Nucleic Acids)

In the device for delivering a nucleic acid of the present invention, the amount of the nucleic acid to be carried on the surface of the solid substrate can be arbitrarily selected depending upon the object of using the device. Usually, however, the amount of the nucleic acid is from 20 nanograms to 10 micrograms, preferably, from 100 nanograms to 5 microgram and, more preferably, from 300 nanograms to 2.5 micrograms per 1 cm² on the basis of the surface over which the solid substrate comes in contact with the cells when it is being used.

Further, the nucleic acid used in the present invention can, usually have a size as described below.

(i) The double strand DNA can have a length of 10 base pairs to 200,000 base pairs, preferably, a length of 10 base pairs to 50,000 base pairs and, particularly, a length of 1,000 base pairs to 10,000 base pairs irrespective of the straight chain or the cyclic chain;
(ii) The single strand DNA (inclusive of the phosphorothioate type) can have a length of 10 bases to 1,000 bases and, preferably, a length of 10 bases to 50 base pairs;
(iii) The double strand RNA can have a length of 10 base pairs to 5,000 base pairs and, preferably, a length of 10 base pairs to 50 base pairs; and
(iv) The single stranded RNA can have a length of 10 bases to 50,000 bases, preferably, a length of 10 bases to 5,000 bases and, particularly preferably, a length of 20 bases to 1,000 bases.

In the present invention, it is allowed to use any nucleic acid having the above-mentioned size depending upon the object of delivering the nucleic acid into the cells. When, for example, it is attempted to treat a disease of a human or any other animal, there is no particular limitation on the nucleic acid that is used if it is a functional nucleic acid that is used for a gene therapy. In this case, preferred examples include plasmid DNA, antisense oligonucleotide, aptamer, ribozyme, siRNA, etc. Among them, a low molecular functional nucleic acid such as siRNA is particularly desired since it does not directly act upon the genome.

In the present invention, further, the above-mentioned nucleic acid can be carried on the surface of the solid substrate in the form of a complex with a polyamine or a cationic lipid.

As the polyamine, there can be exemplified protamine, polylysine, polyethyleneimine, dendritic polylysine and polyamideamine dendrimer. Among them, the protamine is particularly desired since it is easily available and has low cell toxicity. As the cationic lipid, there can be used dioleoyloxypropyl-trimethylammonium (DOTMA), dioleoyl-trimethylammonium propane (DOTAP), and dimethylaminoethanecarbamoyl cholesterol (DC-Chol).

In the above complex, the ratio of the amount of the nucleic acid and the polyamine or the cationic lipid may be selected depending upon the kind thereof so as to obtain a suitable nucleic acid delivery efficiency. In the present invention, it is particularly desired to carry a complex of the nucleic acid and the polyamine. From the standpoint of maintaining a good nucleic acid delivery efficiency, in particular, it is most desired to use a complex in which the polyamine and the nucleic acid are mixed in amounts of such a ratio that the ratio of cation/anion (C/A ratio) is not smaller than 4. If the C/A ratio is smaller than the above range, the nucleic acid delivery efficiency may drop.

### (Carrying the Nucleic Acid)

In the device for delivering a nucleic acid of the present invention, any method can be used for carrying the nucleic acid or a complex of the nucleic acid and a polyamine or a cationic lipid (nucleic acid complex) on the surface of the solid substrate without particular limitation provided it is capable of fixing the nucleic acid or the nucleic acid complex to the surface of the solid substrate. Usually, the solid substrate is dipped in a solution or a dispersion containing the nucleic acid or the nucleic acid complex, is pulled up and is dried. Or, the above solution or dispersion is sprayed onto the solid substrate and is dried. It is further allowable to use a method according to which the above solution or dispersion is dripped onto the solid substrate and, thereafter, an excess of solution or dispersion that is not adsorbed and held, is removed. There is no particular limitation on the solvent or on the concentration of the solution used here; i.e., they can be suitably selected within a range in which the above operation can be carried out. For example, the concentration of the nucleic acid in the above solution or dispersion is from 100 nanograms/ml to 50 μg/ml, preferably, from 500 nanograms/ml to 25 μg/ml and, more preferably, from 1.5 μg to 12.5 μg/ml though it may vary depending upon the kind of the nucleic acid that is used and the solvent for dilution. As required, further, the solution or the dispersion may contain an auxiliary component such as a polymer or a micellating agent for assisting the solid substrate to carry and fix the nucleic acid or the nucleic acid complex.

### (Delivering the Nucleic Acid)

To deliver the nucleic acid into the cells by using the device for delivering the nucleic acid of the invention fabricated as described above, the surface of the device carrying the nucleic acid is simply contacted to, and pressed onto, the cells. The pressing force is such that the predetermined delivery efficiency is maintained within a range in which the cells are not destroyed and is, usually, 5 to 500 g/cm², preferably, 10 to 100 g/cm² and, more preferably, 15 to 50 g/cm². The time for maintaining the pressing is, usually, about 10 minutes to about 3 hours and, desirably, from 20 minutes to 40 minutes. This, however, is not to exclude that the device for delivering the nucleic acid is used depending upon the object of use, and is left to stay in the living body for extended periods of time.

The device for delivering the nucleic acid of the present invention can be fabricated inexpensively and simply. Further, the method of delivering the nucleic acid of the invention using the above device features excellent reproducibility, is easy to operate, and is capable of efficiently delivering the nucleic acid into the cells by pressing the nucleic acid-carrying surface of the device onto the cells exerting a decreased burden on the cells. The solid substrate carrying, for example, plasmid DNA, antisense oligonucleotide or siDNA is capable of delivering the nucleic acid into the cells maintaining good reproducibility and easily. Further, a glass plate can be directly used as the solid substrate without requiring any particular device or reagent that was needed in the conventional gene delivery technology. Stress is given to the cells only temporarily. Upon removing the solid substrate after pressed onto the cells, however, no chemical substance is left at all. Therefore, small burden is given to the cells, and the nucleic acid is delivered highly efficiently.

The device for delivering a nucleic acid of the present invention serves as a fundamental technology for delivering a nucleic acid into the cells and leads to realizing a highly functional device for delivering a nucleic acid that could not be obtained so far.

### EXAMPLES

The invention will now be concretely described by way of Examples to which only, however, the invention is in no way limited.

### Example 1

### Taking the Nucleic Acid Carried on an SEAM-F Coated Substrate into the Cells

A slide glass was cut into a size of 10×10 mm, and was washed with methanol. A 10% SEAM-F/toluene solution was applied in an amount of 100 μL onto the glass substrate, and was heated on a heated block maintained at 50° C. for 5 hours to coat the glass substrate with the SEAM-F.

The SEAM-F used here was the one prepared by blending a pentacopolymer of TMSPMA (10.6 wt %)/MMA (52.8%)/BMA (15.5 wt %)/HEMA (5.3 wt %)/St (15.8 wt %), a polyurethane having a trimethoxysilyl group at both terminals (urethane: 88.6 wt %, aminopropyltrimethoxysilane: 11.4 wt %), and a tetracopolymer of TMSPMA (38.9 wt %)/MMA (2.5 wt %)/PFDEMA (30.3 wt %)/St (28.3 wt %) at a weight ratio of 54:43:3.

Protamine was mixed into 2.5 μg of plasmid DNA such that the C/A ratios were 0, 1, 2, 4, 8 and 16 in 200 μL of sterile water, followed by slight stirring. The mixture was, thereafter, left to stand still for 15 minutes to form a protamine-plasmid DNA complex.

The plasmid DNA was the one prepared as described below.

A luciferase cDNA of a PGL3-control vector (Promega, Madison, Wis., USA) was cut out with a restriction enzyme HindIII/Xbal, and its segment was incorporated into a multicloning site of a pcDNA3 vector (Invitrogen, Carlsbad, Calif., USA). This plasmid (pCMV-Luc) was propagated with a bacillus colibacillus DH5alpha and was refined with the Qiagen Plasmid Giga Kit (QIAGEN GmbH, Hilden, Germany).

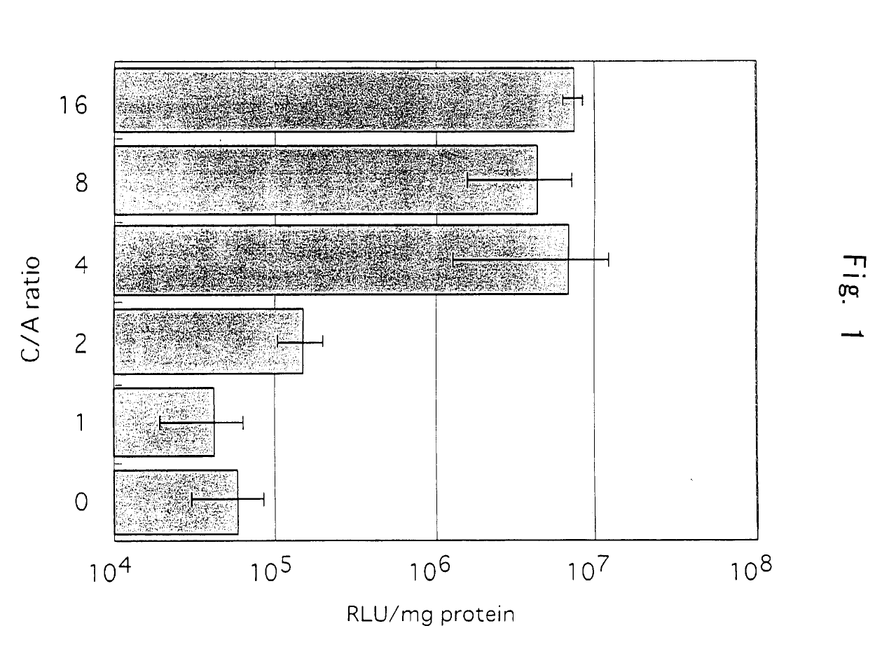

A protamine-DNA complex solution in an amount of 200 μL was placed on the SEAM-F-coated glass so as to be adsorbed thereby for one hour, and was rinsed with sterile water. The obtained SEAM-F-coated substrate carrying the nucleic acid was pressed onto the cultured cells (cells CHO stemming from the ovary of a Chinese hamster) from the upper direction for 30 minutes (with a pressing force of 40 g/cm²) and, thereafter, the substrate was removed. Expression of a gene (luciferase) from the plasmid DNA taken into the cells after 24 hours have passed was evaluated. The results were as shown in FIG. 1.

In FIG. 1, the abscissa represents the amount of expression of the luciferase gene, i.e., represents the amount the gene has worked in the cells, and the ordinate represents the ratio of cations and phosphoric acid groups (anions) in the mixed protamine (C/A ratio). A high expression of gene is recognized when the C/A ratio is not smaller than 4, and the expression becomes stable as the C/A ratio increases.

### Example 2

### Taking the Nucleic Acid Carried on a Glass Substrate into the Cells

The protamine was mixed into 2.5 μg of the plasmid DNA in 200 μg of sterile water such that the C/A ratios were 0, 1, 2, 4, 8 and 16. The mixture was lightly stirred and was left to stand still for 15 minutes. The obtained protamine-plasmid DNA complex in an amount of 200 μm was placed on a glass measuring 10×10 mm so as to be adsorbed thereby for one hour, and was rinsed with sterile water to thereby prepare a nucleic acid-carrying glass substrate.

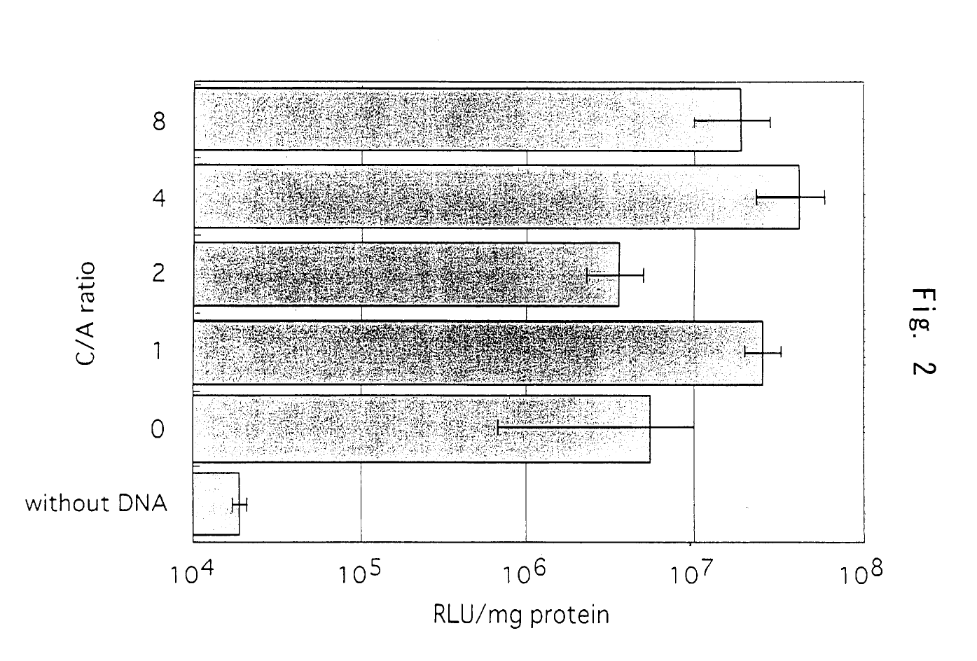

The nucleic acid-carrying glass substrate was pressed onto the CHO cells from the upper direction for 30 minutes (with a pressing force of 30 g/cm²) and, thereafter, the substrate was removed. Expression of a gene (luciferase) from the plasmid DNA taken into the cells after 24 hours have passed was evaluated. The results were as shown in FIG. 2.

From the results of FIG. 2, the expression of the gene was recognized even when the DNA only was adsorbed by the glass substrate (when the C/A ratio was 0). It was, further, learned that a stable and high expression efficiency was exhibited when a complex of the DNA and the protamine was adsorbed.

Further, the protamine was mixed into 5 μg, 2.5 μg, 1.25 μg, 0.625μ and 0.313 μg of the plasmid DNA in 200 μL of sterile water such that the C/A ratio was 4. The mixtures were lightly stirred and were left to stand still for 15 minutes.

The obtained protamine-plasmid DNA complex solutions each in an amount of 200 μL were placed on the glasses measuring 10×10 mm so as to be adsorbed thereby for one hour, and were rinsed with sterile water to thereby prepare a nucleic acid-carrying glass substrates.

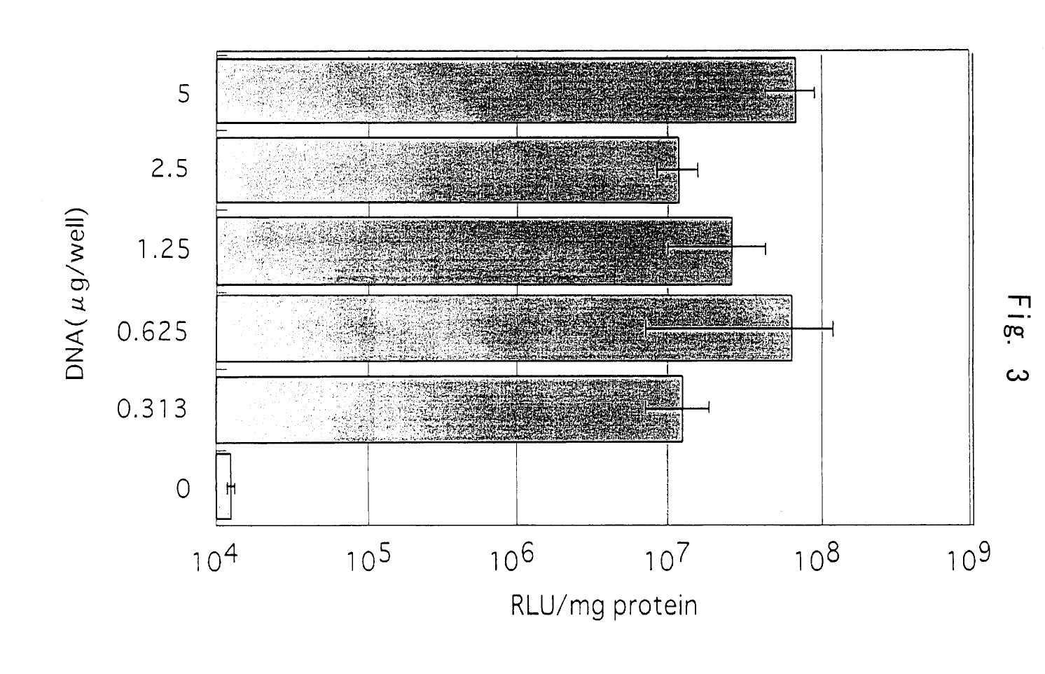

The nucleic acid-carrying glass substrates were pressed onto the CHO cells from the upper direction for 30 minutes in the same manner as described above and, thereafter, the substrates were removed. Expression of genes from the plasmid DNA taken into the cells after 24 hours have passed was evaluated. The results were as shown in FIG. 3.

In FIG. 3, the abscissa represents the amount of expression of the luciferase gene and the ordinate represents the amount of DNA added per a well of a culturing laboratory dish. From the results of FIG. 3, a high expression of the gene was already recognized with 0.313 μg, and it was learned that the gene could be delivered to a sufficient degree by using the DNA in an amount of at least 0.313 μg.

Further, the plasmid DNA was mixed in amounts of 5 μg, 2.5 μg, 1.25 μg, 0.625 μg and 0.313 μg into 200 μL of sterile water to obtain protamine-DNA complex solutions. The obtained protamine DNA complex solutions were placed each in an amount of 200 μL on the glasses measuring 10×10 mm so as to be adsorbed thereby for one hour, and were rinsed with sterile water to thereby prepare nucleic acid-carrying glass substrates.

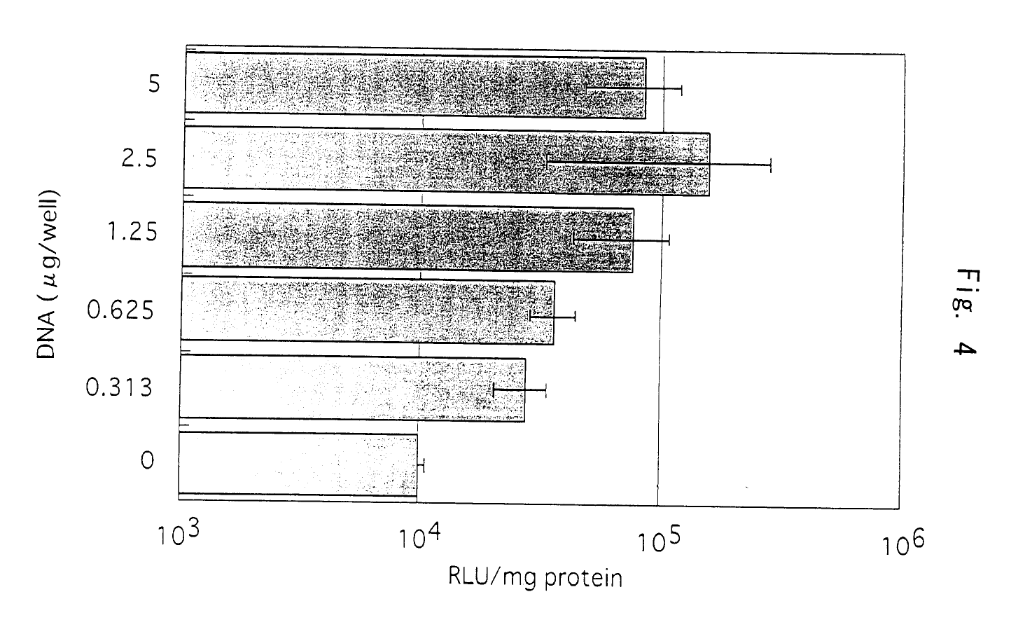

The obtained nucleic acid-carrying glass substrates were pressed onto the CHO cells from the upper direction for 30 minutes in the same manner as described above and, thereafter, the substrates were removed. Expression of genes from the plasmid DNA taken into the cells after 24 hours have passed was evaluated. The results were as shown in FIG. 4.

1. A method for delivering a nucleic acid by pressing the nucleic acid carried on a surface of a solid substrate onto cells to thereby introduce the nucleic acid into the cells.

2. The method for delivering a nucleic acid according to claim 1, wherein the nucleic acid is forming a complex thereof with a cationic lipid or a polyamine.

3. The method for delivering a nucleic acid according to claim 2, wherein the nucleic acid is forming a complex thereof with a protamine or a dendritic polylysine.

4. The method for delivering a nucleic acid according to claim 1, wherein the nucleic acid is a nucleic acid useful for a gene therapy.

5. A device for delivering a nucleic acid in which the nucleic acid to be introduced into cells is carried on a surface of a solid substrate, and the surface of the solid substrate carrying the nucleic acid is pressed onto the cells, so that the nucleic acid is introduced into the cells.

6. The device for delivering a nucleic acid according to claim 5, wherein the surface of the solid substrate is modified with a cationic functional group.

7. The device for delivering a nucleic acid according to claim 5, wherein the surface of the solid substrate is modified with an anionic functional group, and the nucleic acid carried on the surface modified with the anionic functional group is forming a complex thereof with a cationic lipid or a polyamine.

8. The device for delivering a nucleic acid according to claim 5, wherein the nucleic acid carried on the surface of the solid substrate is forming a complex thereof with a protamine or a dendritic polylysine.

9. The device for delivering a nucleic acid according to claim 5, wherein the nucleic acid is a nucleic acid useful for a gene therapy.

10. The device for delivering a nucleic acid according to claim 5, wherein the nucleic acid is carried in an amount of 20 nanograms to 10 micrograms per 1 cm².

11. A method for screening genes by spotting a plurality of known genes or unknown genes on a surface of a solid substrate, pressing the spotted known genes or unknown genes onto cultured cells or onto organ of an animal, and screening the expressions of the genes.

12. A device for introducing a gene by spotting a plurality of known genes or unknown genes onto a solid substrate, pressing the spotted known genes or unknown genes onto cultured cells or onto organ of an animal to screen expressions of the genes or to analyze functions of the genes.

13. The device for a gene therapy comprising the device for delivering a nucleic acid of claim 9.
